(19) United States
(12) Patent Application Publication
(10) Pub. No.: US 2008/0077028 A1
(43) Pub. Date: Mar. 27, 2008

(54) Personal health monitoring and care system

(75) Inventors: Max Schaldach, Berlin (DE); Wolfgang Geistert, Rheinfelden (DE); Erhard Flach, Berlin (DE); Axel Ulbrich, Berlin (DE); Roland Jacobsen, Berlin (DE)

Correspondence Address: BROWDY AND NEIMARK, P.L.L.C.;624 NINTH STREET, NW, SUITE 300, WASHINGTON, DC, 20001-5303, US

(73) Assignee: BIOTRONIC CRM PATENT, Bar (CH)

(21) Appl. No.: 11/527,733

(22) Filed: Sep. 27, 2006

(51) Int. Cl. A61B 5/04

(52) U.S. Cl. 600/509

(57) ABSTRACT

A personal health monitoring and care system includes at least one diagnostic device (2) implanted in a person (1), a data communication device (4) associated to the diagnostic device (2) for communicating relevant personal health data collected by the diagnostic device (2) to an external data information retrieval device (5), which processes and presents said relevant personal health data to the person (1) via information output means (6), wherein the person (1) according to the relevant personal health data perceived from the information output means (6) is able to start a health relevant action.

### BACKGROUND OF THE INVENTION

1. Field of the Invention

The present invention refers to a personal health monitoring and care system comprising at least one diagnostic device implanted in a person and a data communication device associated to the diagnostic device for communicating relevant personal health data collected by the diagnostic device to an external data information retrieval device.

2. Background Art

U.S. Pat. No. 6,735,479 B2 discloses a lifestyle management system which is destined for patients suffering from e.g. cardiologic diseases. These patients use a patient monitoring system based on an implanted medical device (IMD) like a defibrillator, cardioverter, neurostimulator, insulin pump or the like. The IMD collects diagnostic data and sends same via wireless communication means to a so-called home monitoring system. Furtheron a remote expert station is in bidirectional data communication with said at least one implanted device and said home monitoring system.

The concept of the lifestyle management system disclosed in U.S. Pat. No. 6,735,479 B2 is based on the aim that an expert gets knowledge about the physiological conditions of the patient. Accordingly the remote expert station enables doctors and other health care providers to review, monitor current and long-term trends of the patient's health status and dispense clinical care as needed. Inasmuch the known patient monitoring system does not take into consideration the needs of persons in good health who want to get knowledge about their body conditions and take health relevant action depending on internal changes in body conditions or due to external influences like stress, exercise level during sports, extraordinary events and the like. Inasmuch the known patient monitoring system is no real lifestyle management system as the patient is only informed about the basic nature of his implanted device but cannot act himself to change his own body conditions.

### SUMMARY OF THE INVENTION

It is an object of the invention to provide for a personal health monitoring and care system based on which a person has the capability of retrieving and evaluating data about his body based on which the person can start a health relevant action on its own.

This object is achieved by a personal health monitoring and care system according to the invention comprising beyond the at least one implanted diagnostic device and data communication device an external data information retrieval device which processes and presents said relevant personal health data to the person via information output means, wherein the person according to the relevant personal health data perceived from the information output means is able to start a health relevant action.

Due to the invention the personal health monitoring and care system can collect data of e.g. implanted sensors or other diagnostic systems, the data of which is presented to the system user. He has therefore the opportunity to e.g. start certain action like changing his exercising level during jogging by changing the running speed to adapt the heart rate or by reducing exercising weights in a gym to avoid acidity of the muscles. Further health relevant action can be the reducing or enhancing the intake of minerals, pharmaceuticals or nutrients, i.e. magnesium for the muscles or food without lactose or histamine. Furtheron in case the system user has a therapeutical device, like a insulin pump or heart pacemaker he can change the programmed parameters of this device (within limits set by an attending physician), e.g. changing the mean insulin output or initiating a short term insulin output peak. Finally the system user can contact his doctor or coach to discuss the retrieved personal health data.

### BRIEF DESCRIPTION OF THE DRAWING

FIG. 1. shows a schematic diagram of a personal health monitoring and care system.

### DESCRIPTION OF THE PREFERRED EMBODIMENT

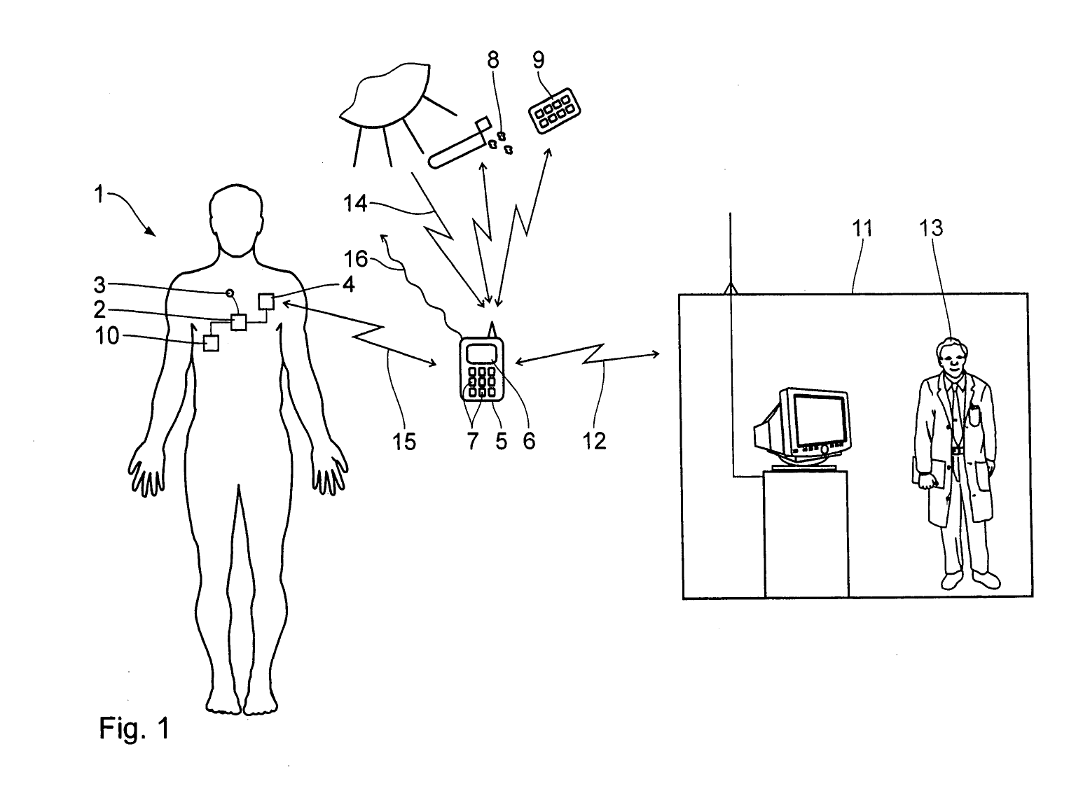

FIG. 1 shows a person 1 who carries a diagnostic device in form of an implant 2. This implant is provided with a sensor 3 which collects data about a physiological parameter, like the blood pressure, heart rate or the like.

The diagnostic implant 2 is connected to a wireless data communication device 4 which communicates the relevant personal health data collected by the implant 2 with the help of the sensor 3 to an external data information retrieval device 5. This may be designed like e.g. a mobile phone including a display 6 and keys 7 to enter instructions or to input data or any other messages.

During the use of the personal health monitoring and care system the person 1 gets health relevant information from the data information retrieval device 5 e.g. by reading an according message in display 6 that the heart rate during jogging exercising is high. It is also possible that the device 5 sends an according audio message 16 to the person. Based upon this information the person 1 can start a health relevant action, e.g. reducing the running speed or dumbbell weight during his exercise. Another health relevant action might be e.g. intaking mineral pills 8 to deliver magnesium to the body of the person 1 or supplemental nutrients 9.

In FIG. 1 there is another implant 10 shown within the body of the person 1 which is a therapeutical implant, like a insulin pump. In case that the person 1 is subject to a higher physical strain the person can decrease the dispensing rate of the implanted insulin pump 10 e.g. by inputting according values via the keys 7 of the data information retrieval device 5.

To assist the person 1 in choosing health relevant action due to the personal health data retrieved from the monitoring and care system a remote expert station 11 is linked to the system. Such remote expert station is to be understood in general terms, i.e. this can be realized by a personal coach, a doctor, a hospital, a health centre, an automatic, computerized expert system or the like. The data information retrieval device 5 is configured to automatically or on demand send the relevant data processed and presented by the data information retrieval device 5 to the remote expert station 11 via a mobile phone or telephone communication line 12. In the remote expert station 11 a doctor or coach 13 evaluates the data and decides whether or not a health relevant action is necessary or at least recommendable. Alternatively the remote expert station 11 contains a computerized expert system, which is able to provide medical advice by means of a large data base and/or artificial intelligence algorithms. The coach/doctor/expert system 13 accordingly can contact the person 1 and give him an appropriate piece of advice to initiate a certain health relevant action. Further on it is possible that the expert, i.e. the doctor or coach 13 or expert system by way of remote control initiates a therapeutical action via the communication line 12, the data information retrieval device 5 and the data communication device 4 at the implant 10 directly, i.e. without involving the person 1.

Finally the data information retrieval device 5 itself can include sensors e.g. for measuring UV radiation 14, environmental temperature, air pressure, ozone or pollen concentration, ingested nutrition or medication, persons weight or the like and to incorporate the according data into the processing of the personal health relevant data.

1. A personal health monitoring and care system comprising:
at least one diagnostic device (2) implanted in a non-expert person (1),
a data communication device (4) associated to the diagnostic device (2) and communicating relevant personal health data collected by the diagnostic device (2) to
an external data information retrieval device (5), which processes and presents said relevant personal health data to the non-expert person (1) via information output means (6),
wherein the non-expert person (1) according to the relevant personal health data perceived from the information output means (6) is able to start a health relevant action.

2. A personal health monitoring and care system according to claim 1, wherein the at least one diagnostic device (2) comprises at least one sensor (3) adapted collecting a relevant physiological parameter.

3. A personal health monitoring and care system according to claim 1, wherein the at least one diagnostic device (2) is combined from or includes a therapeutical device (10) selected from at least one of heart pacemaker, defibrillator, cardioverter, insulin pump or neurostimulator.

4. A personal health monitoring and care system according to claim 3, wherein the therapeutical device (10) comprises an input device (7) to initiate a therapeutical action of the therapeutical device (10).

5. A personal health monitoring and care system according to claim 1, comprising a remote expert station (11) to which the data information retrieval device (5) sends the relevant personal health data.

6. A personal health monitoring and care system according to claim 5, wherein the data communication between the data information retrieval device (5) and the remote expert station (11) is provided via at least one of a wireless, mobile phone or land-line telephone communication means.

7. A personal health monitoring and care system according to claim 5, wherein an expert (13) checks the relevant personal health data and initiates the non-expert person (1) to start a health relevant action.

8. A personal health monitoring and care system according to claim 5, wherein an expert (13) checks the relevant personal health data and by remote control initiates a therapeutical action of a therapeutical device (10) which is combined with or included in the diagnostic device (2) implanted in the non-expert person (1).

9. A personal health monitoring and care system according to claim 7 wherein the expert (13) is a human being or a computerized expert system.

10. A personal health monitoring and care system according to claim 1, wherein the data communication retrieval device (5) communicates via wireless communication line (15) with the data communication device (4) of the diagnostic device (2).

11. A personal health monitoring and care system according to claim 2, wherein the relevant physiological parameter is selected from at least a heart rate, ECG, blood pressure, neuronal potentials or values of physiological fluids.
